(19) United States
(12) Patent Application Publication
(10) Pub. No.: US 2011/0301451 A1
(43) Pub. Date: Dec. 8, 2011

(54) Apparatus And Method For Imaging A Medical Instrument

(75) Inventor: Robert Rohling, Vancouver (CA)

(73) Assignee: The University of British Columbia, Vancouver, BC (CA)

(21) Appl. No.: 13/130,291

(22) PCT Filed: Nov. 24, 2009
(86) PCT No.: PCT/CA2009/001700

§ 371 Date: Jun. 16, 2011

Related U.S. Application Data
(60) Provisional application No. 61/193,390, filed on Nov. 24, 2008.

(51) Int. Cl. A61B 8/00

(52) U.S. Cl. 600/424; 600/443

(57) ABSTRACT

An ultrasound imaging and medical instrument guiding apparatus comprises: a first ultrasound probe configured to acquire a first volumetric dataset representing a 3-D image of a first volume; a second ultrasound probe configured to acquire a second volumetric dataset representing a 3-D image of a second volume; a mount to which the first and second probes are mounted, and a medical instrument guide. The first and second probes are located on the mount such that the first and second volumes overlap to form an overlapping volume. The medical instrument guide is positionable relative to the first and second ultrasound probes and is configured to receive and guide a medical instrument along a propagation axis to a target such that the target and the propagation axis intersect the overlapping volume.

### FIELD OF INVENTION

This invention relates generally to medical imaging, and particular to an apparatus and method for imaging a medical instrument, particularly while being inserted inside a patient.

### BACKGROUND

Some medical procedures require a needle or needle-like instrument to be inserted into a patient's body to reach a target. Examples of these procedures include tissue biopsies, drug delivery, drainage of fluids, ablation for cancer treatment, and catheterization. Some of these procedures can be done manually without any additional guidance other than the sense of feel and visualization of the surface of the body. Other procedures are difficult to perform without additional guidance because the target is deep, the target is small, sense of feel is inadequate for recognizing when the needle's tip has reached the target, or there is a lack of visual landmarks on the body surface. In those cases, providing the health care provider with an image of the interior of the body in the vicinity of the target would be beneficial. It would be particularly beneficial to provide real-time images of both the target and the needle as it progresses towards the target.

A particularly challenging needle insertion procedure is required in epidural anaesthesia, often referred to as an “epidural” in the field of obstetrics. Epidural anaesthesia is administered in the majority (>80% of women in labour) of patients for pain relief of labour and delivery in North American hospitals. Epidural anaesthesia involves the insertion of a needle into the epidural space in the spine. The anatomy of the back and spine, in order of increasing depth from the skin, includes the skin and fat layers, a supraspinous and interspinous ligament, the epidural space, the dura mater and spinal cord. A doctor must insert the needle through these layers in order to reach the epidural space without over-inserting the needle and puncturing the thin dura mater surrounding the spinal cord.

The traditional procedure of epidural needle insertion will now be described. The patient is seated with the doctor facing the patient's back. The doctor chooses a puncture site between the vertebrae based on feeling the protruding spinal processes. After choosing an insertion point on the skin, the doctor typically inserts the needle in a plane midline with the long axis of the spine. A saline-filled syringe is attached to the needle so the doctor can apply pressure to the plunger of the syringe, as the needle in incrementally advanced toward the epidural space, and feel how easily saline is injected into the tissue. In this way, the sense of feel is the main method for determining when the needle tip has reached the epidural space because the saline is easily injected into the epidural space compared to the tissue encountered before the epidural space. This method can result in failure rates of 6 to 20% depending on the experience and training of the health care provider. Complications include inadvertent dura puncture resulting in loss of cerebral spinal fluid and headache, as well as nerve injury, paralysis and even death. Image guidance during needle insertion would improve the accuracy of needle insertion by providing better feedback to the doctor of where the needle is located with respect to the anatomical structures including the target.

In the past several years, ultrasound has been explored as a means to provide a pre-puncture estimate of the depth of the epidural space to correctly place the needle tip. This entails an ultrasound scan prior to needle insertion so that the doctor uses the knowledge of how deep to expect the epidural space when inserting the needle. This use of ultrasound at the planning stage for epidural guidance has received wide interest from the anaesthesia community. It is called pre-puncture scanning because the ultrasound is used before, but not during, needle insertion. The National Institute for Health and Clinical Excellence (NICE) has recently issued full guidance to the NHS in England, Wales, Scotland and Northern Ireland on ultrasound-guided catheterisation of the epidural space (January 2008). While pre-puncture scanning is a useful advance, doctors still face challenges associated with performing needle insertion procedures without information provided by real-time imaging.

There have been some published reports of providing real-time ultrasound imaging for needle insertion procedures. However, none of these approaches have proven to be entirely satisfactory. Problems include overly limiting views of the images of the target and needle due to poor reflection of ultrasound waves, and/or inherent limitations in the ultrasound equipment.

### SUMMARY

It is an object of the invention to provide a solution to at least some of the deficiencies in the prior art.

According to one aspect of the invention, there is provided an ultrasound imaging and medial instrument guiding apparatus comprising: a first ultrasound probe configured to acquire a first volumetric dataset representing a 3-D image of a first volume; a second ultrasound probe configured to acquire a second volumetric dataset representing a 3-D image of a second volume; a mount to which the first and second probes are mounted, and a medical instrument guide. The first and second probes are located on the mount such that the first and second volumes overlap to form an overlapping volume. The medical instrument guide is positionable relative to the first and second ultrasound probes and configured to receive and guide a medical instrument along a propagation axis to a target such that the target and the propagation axis intersect the overlapping volume. Optionally, the apparatus can include a third ultrasound probe that is configured to acquire a 3-D image of a third volume, and which is mounted on the mount such that first, second and third volumes overlap to form the overlapping volume. Either or all probes can be a mechanical 3-D probe or a multidimensional probe. Further, the first and second probes can be curved and/or angled towards the propagation axis.

The mount can be a housing that houses the probes and the medical instrument guide can be a closable channel that extends through the housing between the probes. Or, the mount can be a plate and the medical instrument guide can be a closable channel that extends through the plate between the probes. Or, the mount can be a member and the probes can be mounted to the member such that a space is provided between the probes for location of the medical instrument guide therein. Instead of being permanently affixed to the mount, the medical instrument guide can be detachably mountable to the mount in one or more orientations. Or, the medical instrument guide can be remotely located relative to the probes and can comprise means for tracking the position of the medical instrument guide relative to the probes.

According to yet another aspect of the invention, there is provided a system for acquiring and displaying ultrasound medical images. The system comprises the above ultrasound imaging and instrument guiding apparatus and circuitry that is communicative with this apparatus to receive the first and second volumetric datasets therefrom. The circuitry comprises a processor with a memory having programmed thereon steps and instructions for execution by the processor to: condition the first and second volumetric datasets; combine the first and second volumetric datasets and calculate the overlapping volume in the first and second volumetric datasets; perform one or both of ray-tracing and re-slicing to produce one or more 2-D images from the overlapping volume; and enhance the one or more of the produced 2-D images. The system also comprises a display device communicative with the circuitry to receive and display one or more of the produced 2-D images. The memory can be further programmed to calculate an anticipated trajectory of the needle along the propagation axis, and overlay the calculated anticipated trajectory on one or more of the produced 2-D images. Calculation of the overlapping volume can comprise spatial compounding. Also, ray-tracing can be performed to produce a 2-D projection image of one of the first and second volumetric datasets, or of a combination of the first and second volumetric datasets. Further, re-slicing can be performed on the first or second volumes or the calculated overlapping volume to produce a cross-sectional plane image.

According to another aspect of the invention, there is provided a method of using of the ultrasound imaging and needle guiding apparatus described above in an epidural anaesthetic procedure. The method comprises placing the apparatus over a back of a patient such that the medical instrument guide is placed over a needle insertion point on the back, and emitting an ultrasound signal into the back and capturing images of the first and second volumes, wherein the images of the first and second volumes include a section of a patient's spine. The target can be an epidural space in the patient and each probe can be placed at a paramedian location with respect to the spine, and particularly, over spinae erector muscles of the patient. The method can further comprise inserting the needle through the medical instrument guide and along the propagation axis that intersects the target, such that the captured images includes an image of the needle.

According to yet another aspect of the invention, there is provided an ultrasound imaging and medical instrument guiding apparatus, comprising: a first ultrasound probe configured to acquire a 2-D image of a first plane; a second ultrasound probe configured to acquire a 3-D image of a first volume; a mount on which the first and second probes are mounted, and a medical instrument guide. The first and second probes are located on the mount such that the first volume intersects the first plane. The medical instrument guide is positionable relative to the first and second ultrasound probes and is configured to receive and guide a medical instrument along a propagation axis to a target such that the target and the propagation axis intersect the first volume and first plane.

### BRIEF DESCRIPTION OF DRAWINGS

FIG. 1 is a schematic view of approximate locations of vertebrae, needle puncture point, and various imaging planes of a patient to be imaged by an ultrasound probe and subjected to an epidural anaesthesia procedure.

FIG. 2 is a schematic back view of a dual-probe 3-D ultrasound imaging and needle guiding apparatus according to one embodiment of the invention and positioned to image the spine of the patient shown in FIG. 1.

FIG. 3 is a schematic top view of the ultrasound imaging and needle guiding apparatus and a cross-section of the patient's torso.

FIG. 4 is a schematic back view of the ultrasound probes of the ultrasound imaging and needle guiding apparatus along with a representation of the spatial volume of the ultrasound image captured by each ultrasound probe.

FIG. 5 is a schematic top view of the ultrasound probes of the ultrasound imaging and needle guiding apparatus along with a representation of the spatial volume of the ultrasound image captured by each ultrasound probe.

FIGS. 6(a)-(c) are schematic perspective views of the ultrasound probes of the imaging and needle guiding apparatus and a representation of the spatial volume of the ultrasound image captured by each ultrasound probe, wherein FIG. 6(a) shows rays used in a raytracing imaging technique, FIG. 6(b) shows a cross-sectional re-slice of overlapping volumes in a sagittal plane, and FIG. 6(c) shows a sectional re-slice in a transverse plane.

FIG. 7 is a schematic top view of the probes of the imaging and needle guiding apparatus and a needle, and a representation of ultrasound waves capturing an image of the needle.

FIG. 8 a schematic top view of a pair of curved ultrasound probes of the imaging and needle guiding apparatus according to another embodiment of the invention, along with a representation of the spatial volume of the ultrasound image captured by each ultrasound probe.

FIG. 9 is a block diagram of an imaging system comprising the ultrasound imaging and needle guiding apparatus.

FIG. 10 is a flow chart of a method for processing data from two 3-D images captured by the ultrasound imaging and needle guiding apparatus.

FIG. 11 is a schematic back view of an ultrasound imaging and needle guiding apparatus having one 3-D ultrasound probe and one 2-D ultrasound probe according to another embodiment of the invention, along with a representation of the spatial volume and spatial area of the ultrasound image captured by each respective ultrasound probe.

FIG. 12 is a schematic perspective view of an ultrasound imaging and needle guiding apparatus with three ultrasound probes according to another embodiment of the invention.

FIG. 13 is a schematic view of a display device displaying multiple images captured by the ultrasound imaging and needle guiding apparatus.

FIG. 14(a) is a schematic front view of a detachable medical instrument guide and FIG. 14(b) is a schematic front view of the detachable medical instrument guide attached to an ultrasound imaging and needle guiding apparatus according to another embodiment.

FIG. 15 is a schematic back view of an ultrasound imaging and needle guiding apparatus having a rod mount wherein the space between the ultrasound probes serves as a medical instrument guide according to yet another embodiment.

### DETAILED DESCRIPTION

Ultrasound imaging is a technique for imaging the interior of the body with high frequency sound waves. A standard ultrasound probe comprises a set of transducer elements emitting sound waves into the body. The sound waves reflect on tissue or bone in the body and the reflected sound (echo) is detected by the same transducer elements. By calculating the time from emission to detection of the sound waves at each transducer and measuring the intensity of the reflected sound wave, an ultrasound image can be constructed that shows various anatomical features in the ultrasound probe's field of view.

Ultrasound scanning during a needle insertion procedure enables the observation of both the needle and the target on a real-time ultrasound display. One advantage of such an ultrasound scanning-assisted needle insertion procedure is the ability for the doctor to modify the path of needle insertion to correct the trajectory towards the target. Embodiments of the invention described herein relate to an ultrasound imaging and needle guiding apparatus for guiding a needle to a target in a patient's body, such as the epidural space of the spine, and for acquiring real-time ultrasound images of the needle and target. Specifically, these described embodiments provide real-time or near real-time 3-D images of both the needle and the surrounding tissue and bone of the body using at least two ultrasound probes while the needle is being inserted through a medical instrument guide. At least one of these probes is a 3-D ultrasound probe. In some embodiments, there is an ultrasound imaging and needle guiding apparatus with a pair of 3-D ultrasound probes which are placed in a slightly paramedian position, with one on each side relative to a midline needle insertion position, which enables the ultrasound imaging and needle guiding apparatus to clearly view both the needle and the target, such as an epidural space. In addition, some of the described embodiments include a method for using the ultrasound imaging and needle guiding apparatus and for processing acquired 3-D volumetric datasets from at least two ultrasound probes with intersecting scanning volumes for representation on a 2-D display.

Directional terms such as “top”, “bottom”, “left” and “right” are used in the following description for the purposes of providing relative reference only, and are not intended to suggest any limitations on how any apparatus or components thereof are to be manufactured or positioned during use.

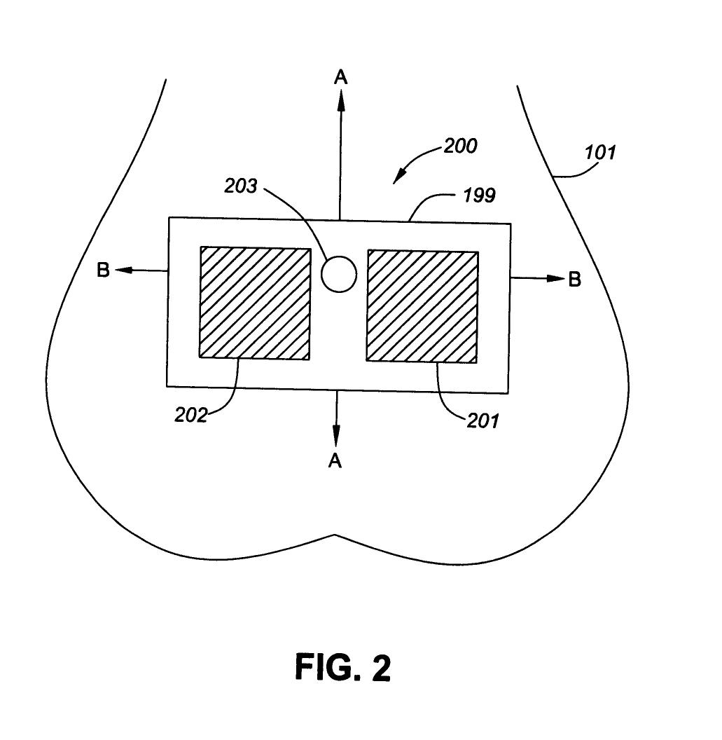

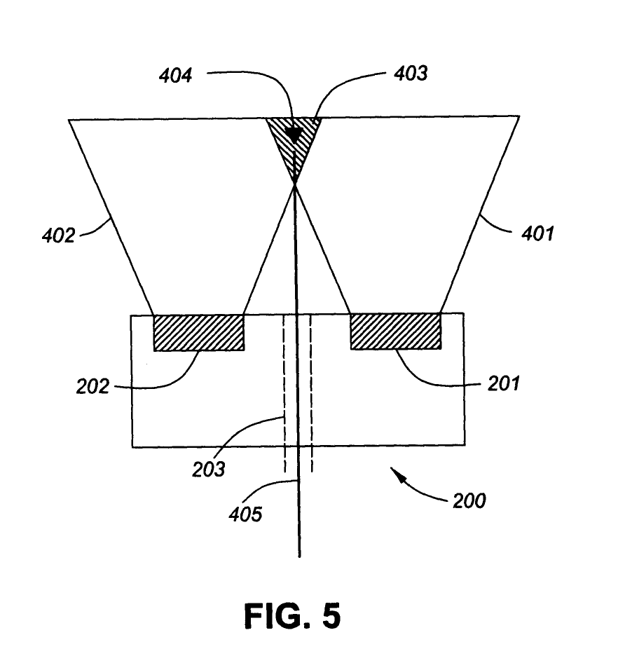

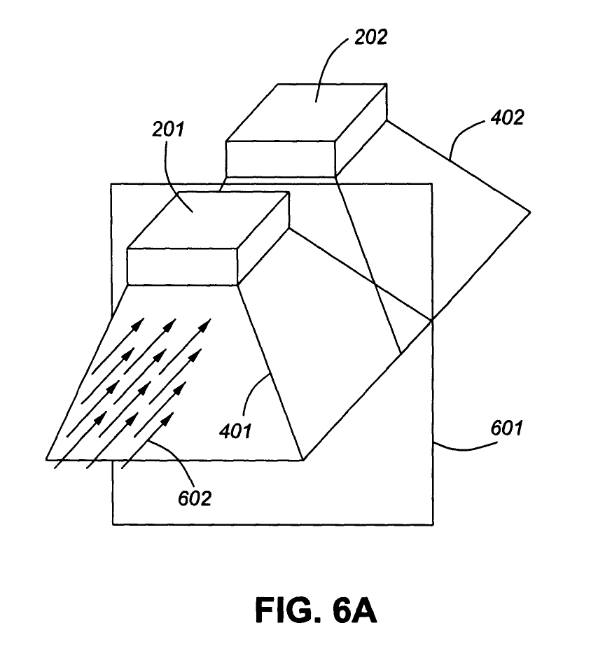

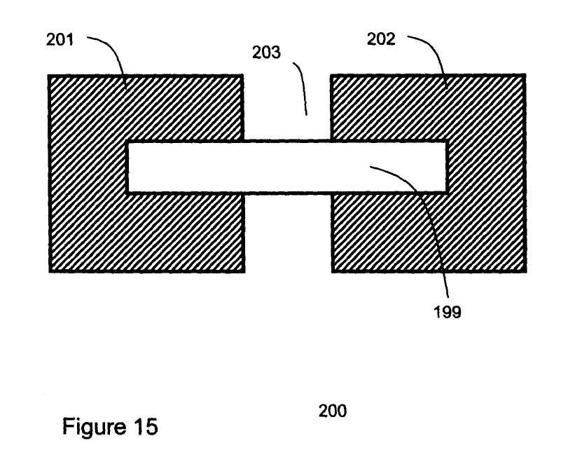

According to a first embodiment and referring to FIGS. 2 to 7, there is provided an ultrasound imaging and needle guiding apparatus 200 that enables the acquisition of simultaneous, or near simultaneous, images of anatomical features of a body 101 and a needle 405 (as shown in FIG. 5), ablation probe, catheter, guide wires or other medical instrument that is inserted into the body 101 and guided by the apparatus 200 and towards a target 404. The main components of the apparatus 200 are two 3-D ultrasound probes 201, 202, a mount 199 on which the probes 201, and a medical instrument guide 203 that in this embodiment is permanently affixed to the mount 199 but in other embodiments can be detachably mounted to the mount 199 or remotely located. The ultrasound probes 201, 202 are spaced from each other and are positioned on the mount 199 to provide simultaneous or near-simultaneous 3-D imaging of a volume of interest in the body 101 and of the medical instrument 405 inserted into the volume of interest. The mount 199 in this embodiment is a housing in which probes 201, 202 are housed; alternatively, the mount 199 can be a rectangular mounting plate (not shown) to which the probes 201, 202 are mounted, or a rod or similar-shaped member to which the probes 201, 202 are mounted (as shown in FIG. 15).

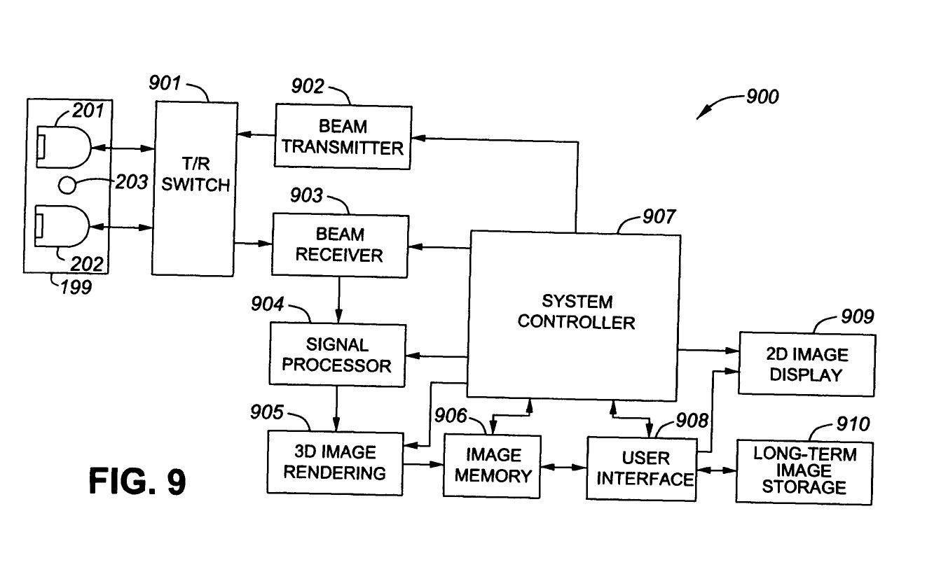

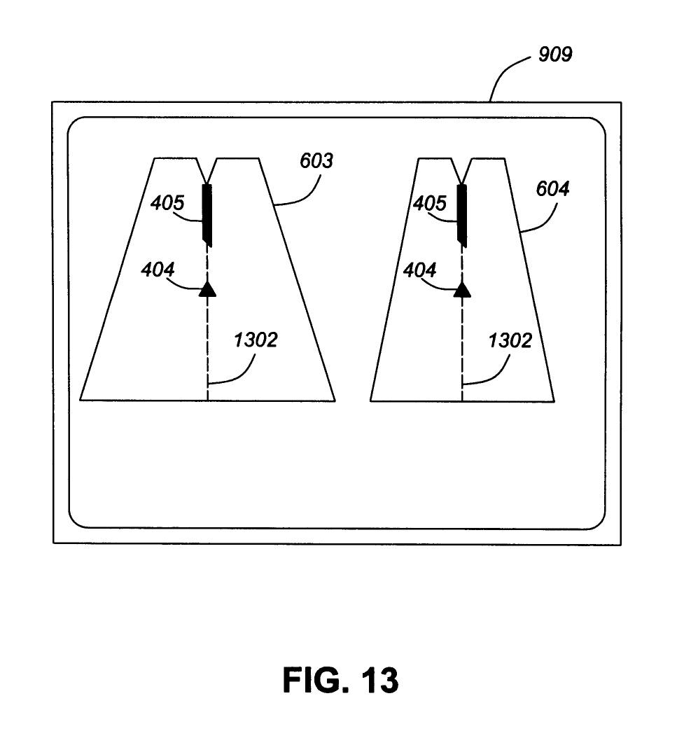

As shown in FIG. 9 and as will be described in more detail below, the apparatus 200 can be coupled to a data processing and display system 900, which includes circuitry 904, 905 for processing volumetric datasets representing the ultrasound images captured by the probes 201, 202, and a display device 909 for viewing the processed ultrasound images. As shown in FIG. 13, the 3-D ultrasound volumetric datasets obtained by the ultrasound probes 201, 202 can be processed and displayed as a single or as multiple image(s) of the volume of interest and the medical instrument 405 inserted into the volume of interest.

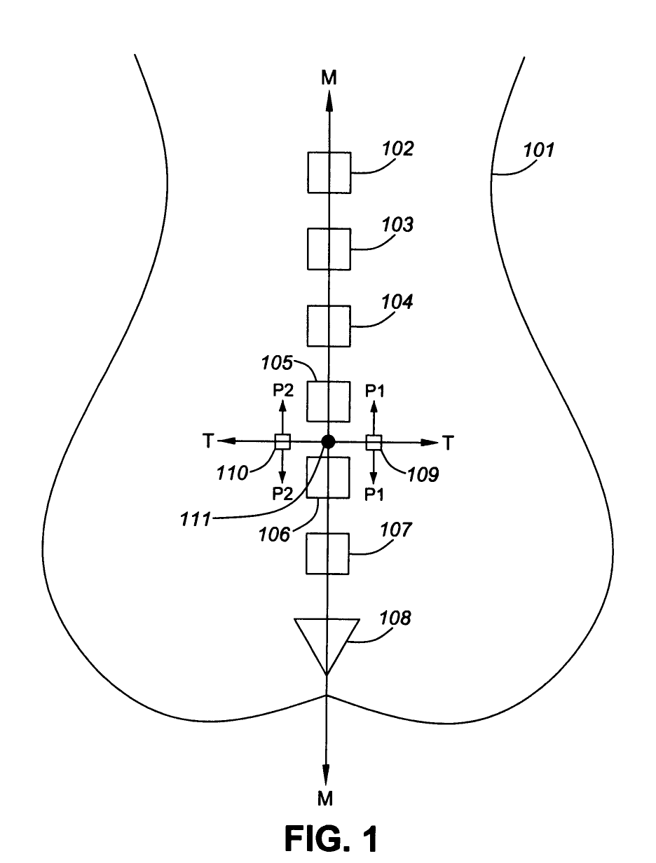

One particular application of this apparatus 200 is for imaging the anatomy of a patient's spine and a needle during an epidural injection, in which case the medical instrument 405 is an epidural needle and the target is the epidural space. FIG. 1 shows the lower back of a patient's body 101; the vertebrae of the lower back are the thoracic vertebrae T12 102, lumbar vertebrae L1 103, L2 104, L3 105, L4 106, L5 107 and the sacrum 108. A preferred needle puncture site 111 is located between the third lumber vertebra L3 105 and the fourth lumbar vertebrae L4 106 in the midline M-M of the patient's spine and along a transverse T-T plane. The apparatus 200 in this application is designed as a portable device that a health care provider can place on the back of the patient undergoing the epidural injection. The apparatus 200 is positioned near the preferred puncture site 111 such that the health care provider may image the back and spine underneath the apparatus 200 and detect in the ultrasound image both the major anatomical features of interest and the tip and body of the needle 405 during the injection. The probes 201, 202 are located on the mount 199 such that when the apparatus 200 is placed on the back of the patient with the medical instrument guide 203 directly above preferred puncture site 111, the probes 201, 202 are located at positions 109, 110 directly above the spinae erector muscles; it is expected that the muscle tissue at these locations 109, 110 serves as “windows” that transmits ultrasound particularly well. The probes 201, 202 can also be oriented on the mount 199 such that propagation of the sound waves from each probe 201, 202 is directed towards the spine. Optionally and not shown, rails can be provide on the mount 199 and the probes 201, 202 can be slidably mounted on these rails to allow for adjustment of the probes' position to suit patients of different sizes.

The back of the mount 199 (i.e. the portion facing away from the body 101 during use) is provided with a hand grip that is shaped and sized to allow for easy single-handed gripping by the operator. Although not shown, the back of the mount 199 can be further provided with finger grips shaped to accept the fingers of the operator. Alternatively, the apparatus 200 is provided with an easy to grasp handle (not shown) so that the operator may hold the apparatus 200 with one hand comfortably against the patient's back during the procedure. The handle may be a basket type handle or pistol-shaped grip protruding from the back of the mount 199.

The two 3-D probes 201, 202 emit sound waves into a 3-D volume that covers the part of the patient's spine underneath the apparatus 200, typically near the L3 and L4 vertebrae. The received data from the reflected sound waves create a volumetric dataset (often abbreviated as “volume”) of the anatomy, unlike a 2-D ultrasound probe which creates images of a cross-sectional plane. The 3-D volume can be viewed by the operator in a number of ways, including a 3-D rendering on the 2-D display device 909 created by ray-casting or ray-tracing techniques adapted from the field of computer graphics, or by re-slicing the volume along a user-defined plane and displaying the cross-section of the volume. The ability to view user-defined slices of the volume at any desired location and angle may be a way to alleviate the limitations of conventional two fixed planes in biplane 2-D probes.

Real-time 3-D ultrasound imaging can be implemented by at least the following two methods:
1) mechanical sweeping: A specialized 3-D probe is constructed by combining a 2-D probe with a motorized mechanism for rapidly moving the 2-D probe so that the 2-D image sweeps repeatedly through a volume of interest. Repeated sweeping is usually implemented in an oscillating manner where each oscillation produces a 3-D volume. The spatial relationship between the set of 2-D images from each oscillation is known because the probe motion is controlled and the images are reconstructed into a 3-D Cartesian volume. This device is referred to hereafter as a mechanical 3-D probe;
2) multidimensional arrays: A specialized probe is created without a motorized mechanism, but instead uses a two dimensional array of transducers to scan over a 3-D volume of interest. The speed of volume acquisition is typically higher than mechanical probes but the complexity of the probe increases and image quality can be inferior. This probe is known as a multidimensional probe.

The 3-D probes 201, 202 of the apparatus 200 can be a mechanical 3-D probe or a multi-dimensional 3-D probe as known in the art. An example of a suitable mechanical 3-D probe is the RAB2-5 H46701 M for the Voluson 730 ultrasound machine by General Electric Corporation (GE Healthcare, Chalfont St. Giles, United Kingdom). An example of a suitable multidimensional probe is the X7-2 for the Philips iU22 ultrasound machine (Philips Healthcare, Andover, Mass., USA). With such types of probes, the rapid creation of 3-D volumes allows multiple planes of the acquired volumes to be visualized in real-time, thus overcoming some of the limitations of standard 2-D probes. These planes can be selected at any orientation and location within the volume through user control.

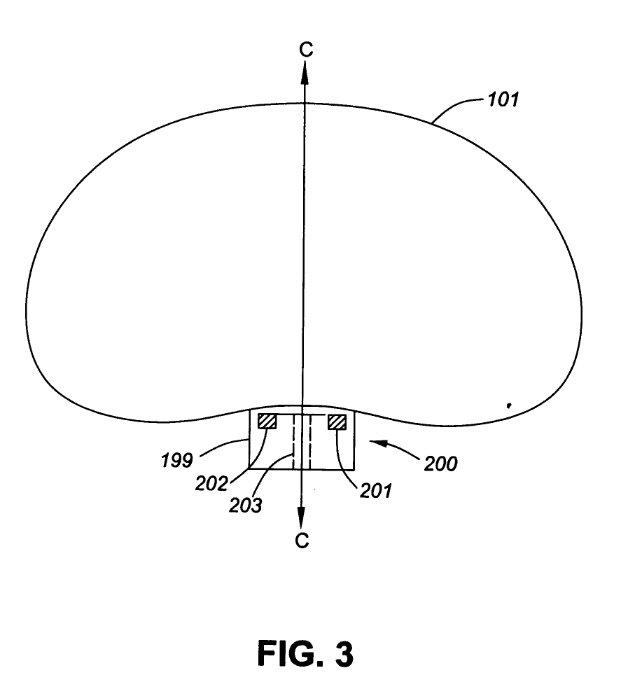

The medical instrument guide 203 in this embodiment is a channel which extends through the mount 199 and is sized to receive the epidural needle 405; although not shown the channel can have a closable cover that extends along part or the entire length of the channel and which can be opened to allow access therein for cleaning etc. The medical instrument guide 203 is positioned between the 3-D probes 201, 202 and is used to constrain the path of the epidural needle 405 inserted during the injection procedure. When the apparatus 200 is placed on the patient's back, the axis A-A (see FIG. 2) of the apparatus 200 is aligned approximately to within 10 to 20 degrees, measured about the axis of the medical instrument guide 203, of the midline axis of the spine M-M (see FIG. 1) in the inferior-superior direction, while the axis B-B of the apparatus 200 is orthogonal to axis A-A and is aligned to extend to the left and right of the patient along transverse axis T-T (see FIG. 1). The axis of the medical instrument guide 203 is aligned approximately with the axis C-C which is the horizontal axis extending through the needle insertion point 111 (see FIG. 3) and is directed towards the patient's back in the anterior-posterior direction. The apparatus 200 can be provided with markings (not shown) representing axes A-A, B-B, and C-C to assist the operator in correctly positioning the apparatus 200 against the patient's back during use.

As will be discussed further below, the apparatus 200 obtains volumetric datasets that are processed by the system 900 and displayed in multiple real-time views which assist the operator in guiding the medical instrument 405 to the target. Two of these views include the sagittal plane which is the plane along axes M-M and C-C and the transverse plane which is the plane along axes T-T and C-C.

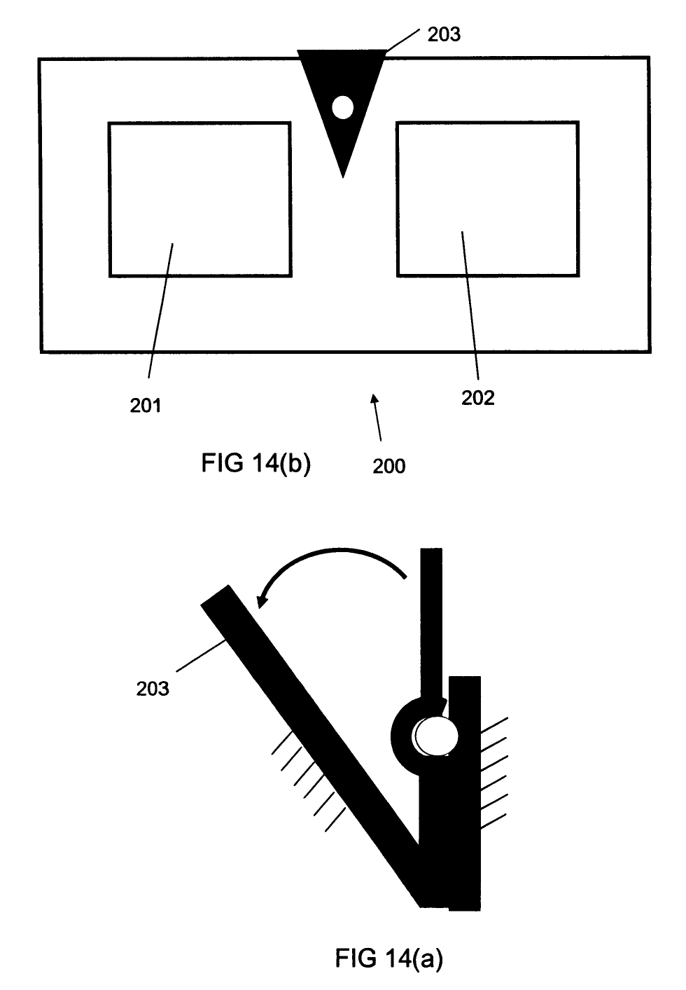

While a channel through the mount 199 serves as the medical instrument guide 203 in this embodiment, the medical instrument guide can be a bore, slot, aperture, hole or any guideway which serves to constrain the path of the needle 404 during the insertion procedure. As shown in FIG. 15, the medical instrument guide is the space between the probes 201, 202 that are interconnected with a rod-shaped mount 199. Also, the medical instrument guide can either be permanently affixed to the apparatus 200 as shown in FIGS. 2-7, or be a separate component which can be detachably mounted to the mount 199 as shown in FIG. 14; in this Figure, the guide 203 is a clip having three members pivotably connected about a pivot axis; the instrument guide 203 can be attached to a channel located between the probes 201, 202 and at the edge of the mount 199. The detachable medical instrument guide can be designed to allow the selection of a particular trajectory to be chosen by mounting one of a series of medical instrument guides, each with a different orientation of the guide-way. The detachable medical instrument guide can also be disposable after a single use for the purposes of ease-of-sterilization.

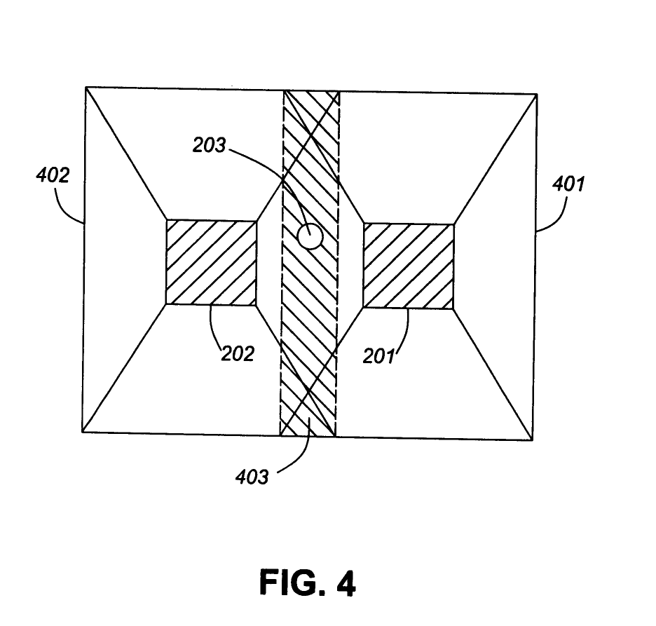

As can be seen in FIG. 4, the probes 201, 202 are positioned and operated so that a portion of the volume 401 produced by probe 201 and a portion of the volume 402 produced by probe 202 overlap to form overlapping portion 403 (shown in cross-hatched shading), which intersects the medical instrument guide 203 and at least part of the pathway of the needle 405 inserted through the guide 203. As can be seen in FIG. 5, the instrument guide (bore) 203 and probe 201, 202 locations are positioned relative to each other so that the overlapping portion 403 covers a target 404 which represents the epidural space, and the part of the needle pathway leading up the target 404. In this Figure, the needle 405 is shown partly inserted into the medical instrument guide 203 in a direction that will intersect the target 404.

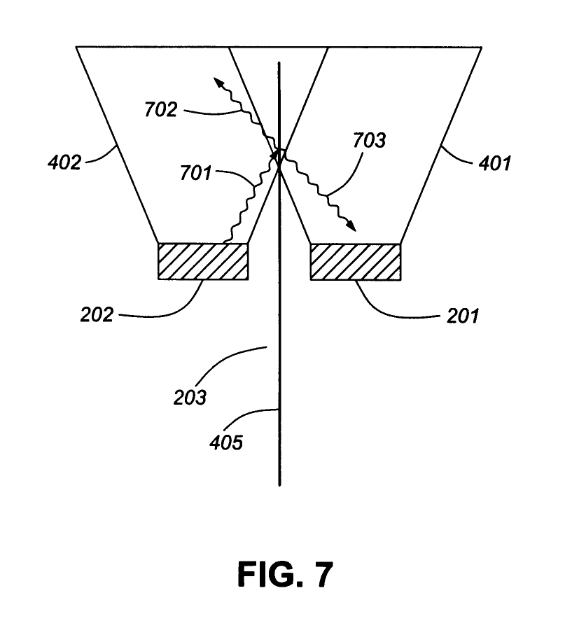

FIG. 7 shows how the needle 405 can be detected by the control of an ultrasound pulse transmission and receive processing. Probe 202 emits an ultrasound beam 701 which reflects off of the needle 405 and creates a reflection 702. Reflection 702 travels away from the needle 405 deeper into the tissue, and also produces subsequent reflections 703 upon further interaction with tissue reflectors such as tissue boundaries. The reflection 702 follows the law of specular reflection from beam 701, meaning that the angle of reflection from the needle 405 equals the angle of incidence of beam 701 to the needle 405. Given knowledge of the direction and timing of beam 701, and the use of the law of specular reflection, the direction of the reflection 702 and subsequent reflection 703, opposite to reflection 702, can be calculated. The subsequent reflection 703 is measured by probe 201 (the reflection can also be measured by probe 202 but the signal will be weaker). The measurements by the probe 201 include one or both of the magnitude and timing of the reflection 703. If the needle 405 is not inserted to a depth that produces an interaction with ultrasound beam 701 then no subsequent reflection 703 is produced. In this way, the measurement of the subsequent reflection 703 provides a measurement of insertion depth of the needle 405 into the tissue. Following this process of producing beam 701 at different source locations and angles, and measurements of the subsequent reflections 703 for each of the different beams 701, the depth of the needle insertion is robustly determined. The measured needle insertion depth is then displayed on the ultrasound display. In another embodiment of the invention, the probe 202 measures the diffuse reflection of ultrasound beam 701 after the beam interacts with the needle 405 to measure the depth of needle insertion. The diffuse reflection produces a wide continuous range of reflections 703 that are measured by probe 202 using one or both of timing and magnitude of the reflections 703. Again, if the needle 405 is not inserted to a depth that produces an interaction with ultrasound beam 701 then no subsequent diffuse reflection is produced. This, in turn, produces a robust measurement of needle insertion depth.

Referring to FIG. 9, an imaging system 900 incorporating the apparatus 200 processes and displays the images obtained by the apparatus 200. In the system 900 shown in FIG. 9, the apparatus 200 is connected to a transmit/receive (T/R) switch 901. The T/R switch 901 receives signals from a beam transmitter 902 and outputs signals to the two probes (201 and 202). The T/R switch 901 also transmits signals from probes 201 and 202 to a beam receiver 903 that forms echo signals for processing. Both the beam transmitter 902 and the beam receiver 903 are communicative with and controlled by a system controller 907. The beam receiver 903 outputs echo signals (representing 3-D volume datasets) from both probes 201, 202 to a signal processor 904, which performs functions such as, but not limited to, digital filtering, contrast detection and enhancement, spectral analysis and B-mode processing; both beam receiver 903 and signal processor are controlled by the system controller 907. Signal processor 904 outputs the modified echo signals to a 3-D image rendering module 905 which converts the 3-D volume datasets into 2-D images using a method such as, but not limited to, reslicing or raytracing. The 3-D image rendering is performed according to instructions provided by the system controller 907, which can receive input from a user interface 908 to determine methodology. 2-D image data sets are transferred into an image memory 906 for access by the user interface 908, for display on a 2-D image display 909 such as a computer screen, and/or for long term storage on a storage device 910 such as a hard drive. The image memory 906 communicates with the system controller 907 and the user interface 908 to access datasets and control filing. The user interface 908 can receive commands from a user to control the operation of the system 900, how image data is processed and displayed on the 2-D image display 909, and to access/store images in the long-term image storage device 910. The user interface 908 includes an interface program that may be integrated with the 2-D image display 909 and may include, but is not limited to, a pointing device such as a mouse or touch screen, a keyboard, or other input devices such as a microphone. The system controller 907 communicates with user interface 908 to relay operational and display instructions and operational status. The system controller 907 communicates with the 2-D image display 909 to synchronize the data stream.

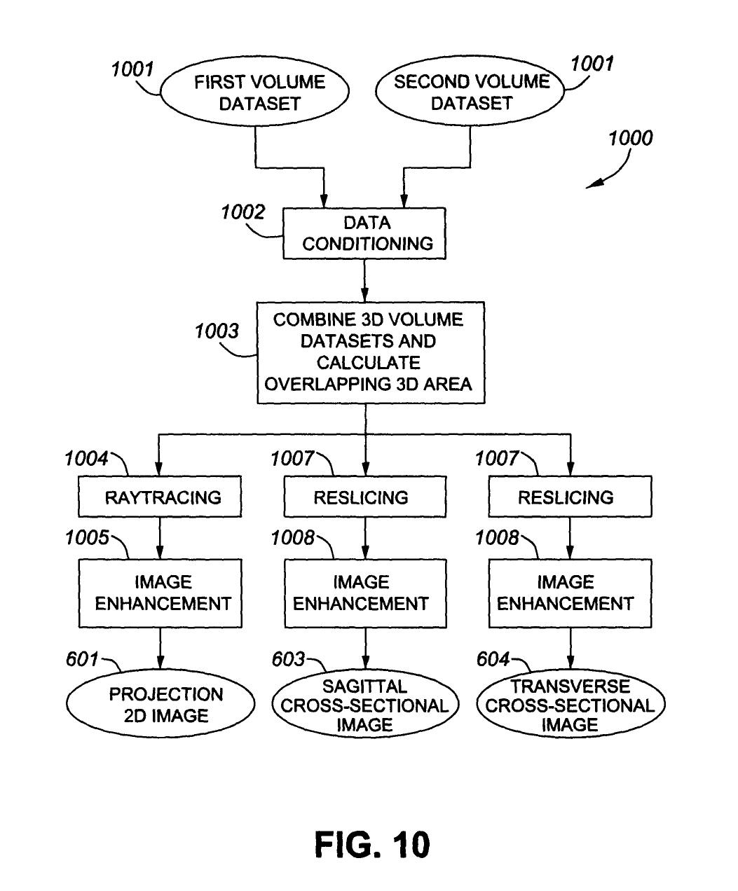

Referring to FIG. 10, a data processing method 1000 is carried out by the system 900 to manipulate the two 3-D image datasets 401, 402 that contain overlapping volumes acquired by the apparatus 200 to produce a 2-D sagittal plane image and a 2-D transverse plane image, which can be displayed on the display device 909. First, the two image datasets 401, 402 are obtained from the apparatus 200 (step 1001) and transmitted via T/R switch 901 and beam receiver 903 to the signal processor 904 for data conditioning (step 1002). Data conditioning performed on the two 3-D image datasets 401 and 402 may include, but is not limited to: filtering, enhancement, thresholding, smoothing and feature extraction. The signal processor 904 also combines the datasets 401 and 402 using their known positions relative to each other and also calculates the overlapping volume 403 (step 1003); the calculation of overlapping 3-D volume 403 on the conditioned dataset may involve spatial compounding to improve image quality or other image processing steps. The combined 3-D image dataset is then transmitted to the image rendering device 905 for projection (step 1004) or cross-sectional (step 1007) image processing.

Instead of a separate signal processor 904, image rendering module 905, controller 907 and memory 906, the steps of the method shown in FIG. 10 can be stored on computer readable medium that can be executed by a general purpose computing device. Examples of suitable computer readable medium are compact disk read only memory (CD-ROM), random access memory (RAM), or a hard drive disk.

When carrying out projection imaging in step 1004, ray-tracing is used to compute a projection of one of the 3-D datasets 401, 402 or a combination of the two datasets 401, 402 in the sagittal plane or on another image plane inputted by the user or automatically selected. Ray-tracing is a popular method for realistically projecting a voxel-based volumetric dataset onto a 2-D image. Ray-tracing involves projecting rays perpendicularly from every pixel in the plane of the 2-D image through the voxels of the volume and calculating for each pixel a value that represents the projection of the voxel values encountered along the corresponding ray.

To perform ray-tracing, the voxel values along the ray path are combined in a variety of ways to derive the pixel value in the projected 2-D image. Examples of how the projected value is calculated from the voxels along the ray path are as follows: (1) The minimum voxel value is chosen. (2) The maximum voxel value is chosen. (3) The average or sum of the voxel values is calculated. (4) The voxel values are weighted according to specific parameters controlling the rendering style, such as a modifying parameter based on a local gradient. (5) Voxel values below a noise threshold are first removed and then the minimum voxel value is chosen. (6) Voxel values below a noise threshold are first removed and then the maximum voxel is chosen. (7) Voxel values below a noise threshold are first removed and then the average or sum of the voxel values is calculated. (8) Voxel values below a noise threshold are first removed and then weighted according to specific parameters controlling the rendering style.

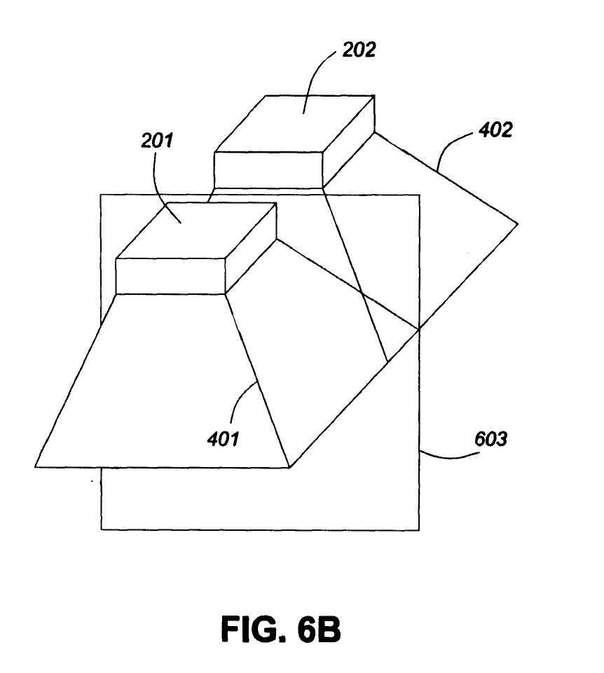

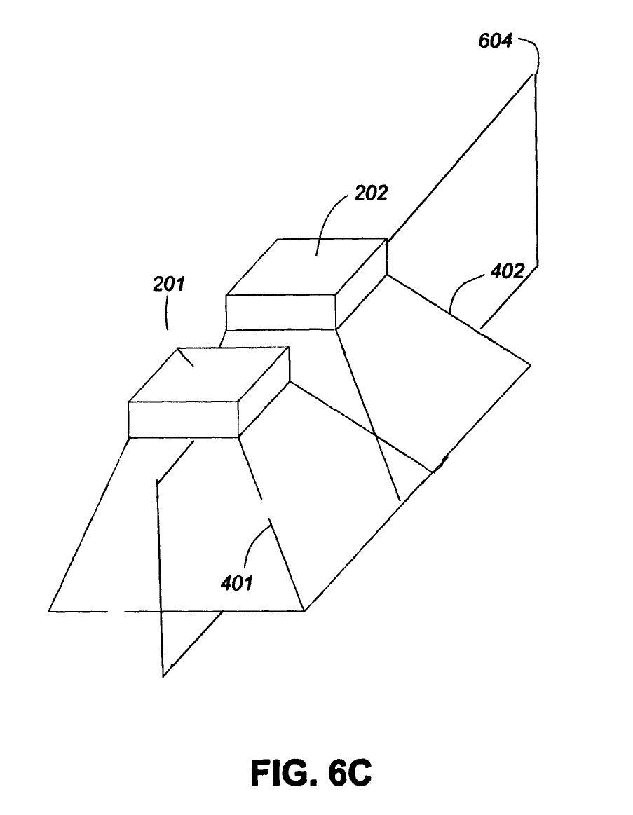

FIG. 6(a) shows how a 2-D image 601 in the sagittal plane can be produced by acquisition of the volumetric dataset (“volume”) 401 from the probe 201, followed by projection of the data of volume 401 through the process of ray-tracing along the rays 602. In this way, the data represented by volume 401 is projected onto the image 601. The process of ray tracing can take the form of averaging of all data encountered in the volume 401 from a single ray 602. The process of ray tracing can also take the form of taking the maximum value of the data encountered along a single ray. Other versions of ray tracing can also be used, including methods that first extract the anatomical features of interest, such as the epidural space, and only project that data onto the image 601. In another embodiment, the ray-tracing can be performed on the combination of the data of volumes 401 and 402.

The resultant 2-D projection image is then processed by the rendering device 905 for image enhancement 1005 which may include, but is not limited to, filtering, enhancement, thresholding, smoothing and feature extraction and results in the final image. In particular, an anticipated needle trajectory can be superimposed onto the projection image. The location of the overlaid trajectory is known and fixed relative to the probes 201 and 202, because it is determined by the physical location of the medical instrument guide 203 on the apparatus 200. The enhanced image is then ready for display by display device 909, and/or storage on storage device 910.

When carrying out cross-sectional image processing, re-slicing (step 1007) is used to produce a 2-D slice of the 3-D image dataset at the transverse plane or sagittal plane (which are the planes that intersect the medical instrument guide for needle insertion). This 2-D slice image is then processed at step 1008 for image enhancement which may include, but is not limited to, filtering, enhancement, thresholding, smoothing and feature extraction and results in the final 2-D sagittal cross-sectional plane image 603 or transverse cross-sectional plane image 604; like the sagittal plane projection image, an anticipated needle trajectory can be superimposed onto the cross-sectional plane image.

Referring now to FIGS. 6(b), 6(c) and 13, the 3-D volumetric datasets measured by the probes 201, 202 are processed by the system 900 and displayed on one or more of the cross-sectional or projection images on a display device 909. The transverse plane image 604 is formed by combining the volumetric datasets 401, 402 then re-slicing, using a method of data interpolation, the combined volumes in a plane that is transverse to the patient and intersects the trajectory 1302 of the medical instrument guide 203. This transverse plane, in which image 604 is formed, can be the same plane as shown FIG. 3. As the operator inserts the needle 405 into the tissue, the needle 405 becomes visible in the image 604, and will be along a graphic overlay 1302 of the expected needle trajectory. As the needle 405 is inserted deeper into the tissue, more and more of the needle becomes visible in the image 604. The operator aligns the needle trajectory 1302 with the target 404 so that subsequent insertion of the needle 405 into tissue reaches the target 404. This image 604 is updated on the display device 909 as the ultrasound 3-D volumetric datasets 401 and 402 are created by probes 201 and 202. In this way, the apparatus 200 provides current images of the needle insertion procedure.

Similar steps as described above can be applied to produce the sagittal cross-sectional re-slice plane image 603.

The projected image 601 can also be shown on the monitor 909. The projected image 601 is formed by projecting the 3-D ultrasound dataset onto a 2-D plane, such as the sagittal plane 601 depicted in FIG. 6(a). The projected image 601 also contains a graphic overlay 1302 of the expected needle trajectory that is also projected in the sagittal plane. The projected image 601 also depicts the needle 405 and the target 404. This image 601 is also updated as the ultrasound 3-D volumetric datasets 401 and 402 are created by probes 201 and 202.

### Operation

In performing an epidural anaesthesia procedure on a patient using the apparatus 200, an operator holds the apparatus 200 with one hand gripping the handle and places the apparatus 200 against the patient's back so that the medical instrument guide 2003 is directly over the needle insertion point 111. The operator then activates the apparatus 200 to cause ultrasound signals to be emitted by the proves 201, 202 and consequent data to be collected and processed by the system 900 and displayed as 2-D images on the display device 909. The two ultrasound probes 201, 202 may be operated alternately, one after the other, so that the sound fields do not interfere. The operator aligns the displayed target (e.g the epidural space) with the superimposed anticipated needle trajectory in the ultrasound image(s). The operator can then insert the epidural needle 405 through the medical instrument guide 203. The operator may then view in real time on the display device 909 a processed ultrasound image of the needle tip and needle body and the patient's back and spine, such as the two images of the sagittal and transverse planes as shown in FIG. 13. The operator may then determine, by viewing the relative motion of the needle tip with respect to the spinal anatomy, when the needle has reached the epidural space of the spine.

As can be appreciated from the above discussion, one advantage of this apparatus 200 is the ability to capture an image of the target, nearby anatomy, and needle trajectory for display in the same display device. Another advantage is the ability to acquire more than one image of the target, nearby anatomy and needle trajectory through the use of two or more 3-D ultrasound probes. Yet another advantage is the ability to use the optimal locations on the skin surface, also known as “windows”, for viewing the spine with ultrasound. Yet another advantage is the ability to place the needle through the medical instrument guide 203 near the middle of the apparatus 200 so that the footprint of the apparatus 200 does not interfere with the puncture site of the needle 405. Yet another advantage is the ability to transmit ultrasound beams from one probe of the apparatus 200 and receive the resulting ultrasound echoes with another probe of the apparatus 200.

### Other Alternate Embodiments

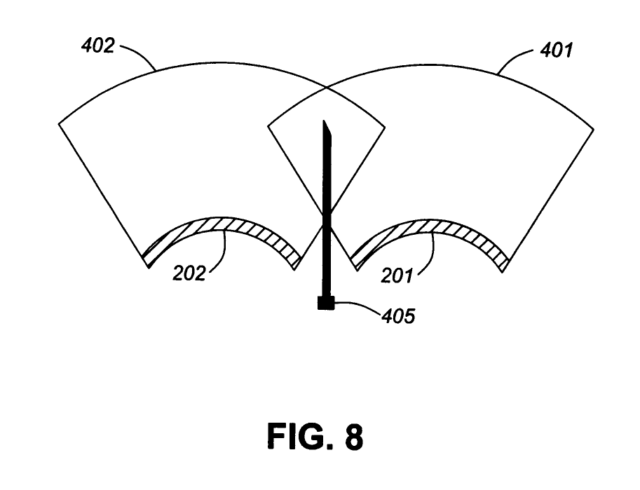

Referring to FIG. 8 and according to another embodiment, the probes 201, 202 are curved. In this embodiment, the size and shape of the 3-D volumes 401 and 402 are determined by the curved shape of the probes 201 and 202. This embodiment has the advantage of obtaining a wide field of view of the anatomy with a relatively small footprint of the probe due to the curvature of the face of the probe that produces a diverging set of beams that are used to form a 2-D image or 3-D volumetric dataset. This embodiment also has the advantage of directing the ultrasound beams toward the needle 405 at an angle that is closer to perpendicular to the needle 405, resulting in a stronger echo from the needle 405 and a better depiction of the needle 405 in the 3-D volumetric dataset.

In yet another embodiment (not shown), the probes 201, 202, whether flat or curved, can be further angled toward each other so that the beams intersect the needle at angles even closer to perpendicular.

The apparatus 200 may also be used for the purposes of tissue tracking for elastography. Probe 201 emits an ultrasound beam which encounters a moving portion of tissue. The motion can be measured from the echo signals of that beam using elastography techniques. Similarly, the motion can be measured by a beam from probe 202. Each probe 201, 202 can measure different components of the tissue motion with different levels of accuracy depending on the orientation of the beam with respect to the tissue motion. Typically motion in the direction of the beam is most accurate. The use of the different components of motion can then subsequently be used in an elastography system to produce estimates of the tissue mechanical properties. The use of three probes allows all three directions of the motion to be estimated in 3-D space.

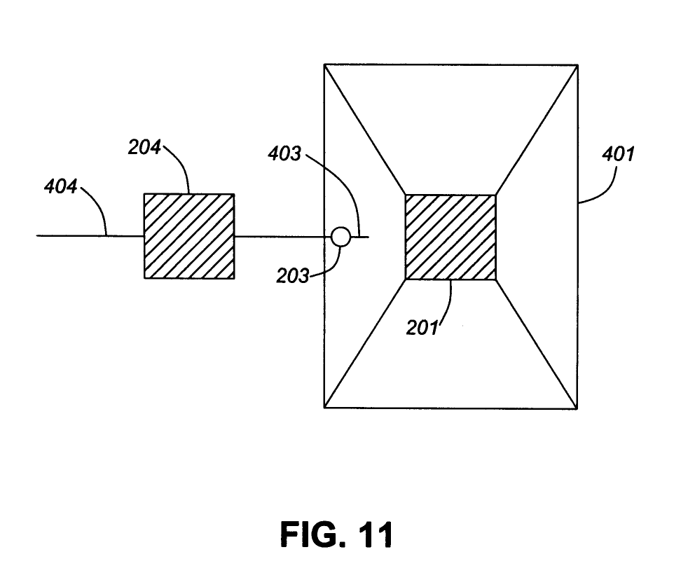

FIG. 11 shows another embodiment of the invention wherein the apparatus comprises at least one 3-D probe 201 and at least one 2-D probe 204. The 3-D ultrasound probes 201 creates a 3-D volumetric dataset 401 and the 2-D probe 204 creates a 2-D cross-sectional image 404. The 2-D cross-sectional image is oriented in a direction that intersects the trajectory of the needle defined by the medical instrument guide 203. The 2-D cross-sectional image also intersects the 3-D volumetric dataset 401. In this way there is still a region of overlap 403 (not emphasized in figure for clarity) between the volumetric dataset 401 and the image 404 that is suitable for spatial compounding. In this embodiment, the combination of a 3-D probe 201 and 2-D probe 204 is advantageous when faster image formation is desired, since the speed of creating a 2-D image 404 is often faster than the speed of creating a 3-D volumetric dataset 401.

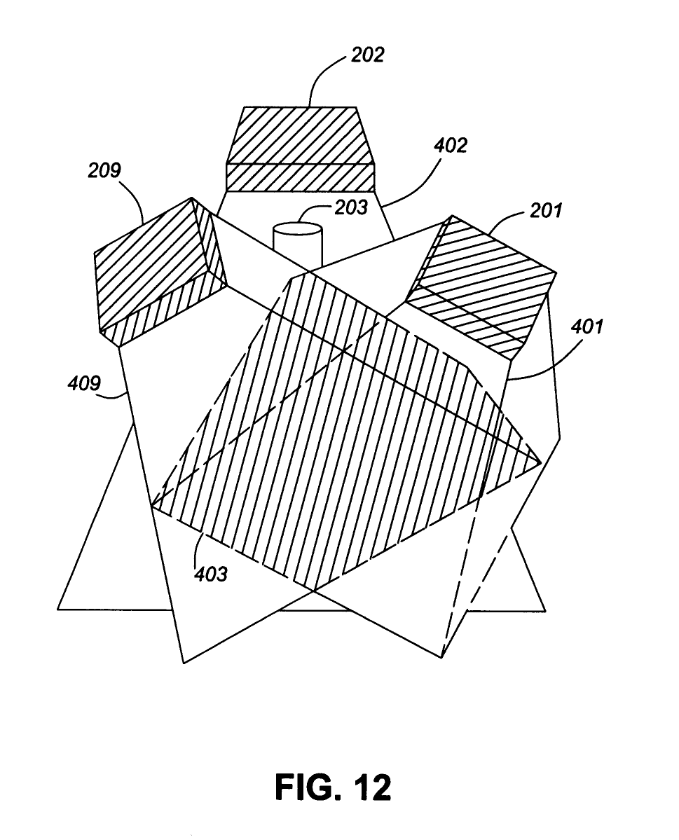

FIG. 12 shows another embodiment of the invention wherein the apparatus 200 comprises three ultrasound probes: probe 201 producing 3-D volumetric dataset 401, probe 202 producing 3-D volumetric dataset 402 and probe 209 producing 3-D volumetric dataset 409. The three probes 201, 202, 209 and aligned and operated so that the three volumes 401, 402, 409 overlap; the overlapping portion 403 is the volume of the intersection of the 3-D volumetric datasets 401, 402 and 409. The expected needle trajectory, defined by the medical instrument guide 203, passes through the overlapping portion 403. When three or more probes are used to create 3-D volumetric datasets, three or more images can be formed and displayed on the monitor 909. In another embodiment (not shown), one or more of the 3-D volumetric datasets is replaced with a 2-D cross-sectional image, such as shown in FIG. 11. In another embodiment (not shown), the sagittal cross-sectional image 603 is replaced by a sagittal projection image 601.

According to yet another embodiment (not shown), the needle guide 203 is not permanently or detachably mounted to the mount 199 and instead is a component of the apparatus 200 that is located remotely of the probes 201, 202 and mount 199. Both the probes 201, 202/mount 199 and needle guide 203 are provided with a position tracking system that provides measurements of the needle location and orientation relative to the ultrasound probes. The tracking system can be based on electromagnetic tracking of coils placed on both the needle 405 and the apparatus 200. A tracking system can also be based on optical tracking of visible fiducials placed on both the needle 405 and the apparatus 200. Furthermore, a tracking system can be based on a moveable needle guide connected to the apparatus 200 by one or more linkages with angle sensors on the linkage joints. With any such needle position tracking system, the expected needle trajectory can be calculated from the measured needle location and orientation. This expected needle trajectory can be shown as a graphic overlay 1302 on any of the images 601, 603 or 604. In use, the operator can position the needle guide 200 such that the propagation axis of the projected trajectory will fall within the overlapping volumes 401, 402 of the probes 201, 202 and thus be displayable on the display device 909.

1. An ultrasound imaging and medical instrument guiding apparatus, comprising:
a first ultrasound probe, configured to acquire a first volumetric dataset representing a 3-D image of a first volume;
a second ultrasound probe, configured to acquire a second volumetric dataset representing a 3-D image of a second volume;
a mount to which the first and second probes are mounted;
the first and second probes located on the mount such that the first and second volumes overlap to form an overlapping volume; and
a medical instrument guide positionable relative to the first and second ultrasound probes and configured to receive and guide a medical instrument along a propagation axis to a target such that the target and the propagation axis intersect the overlapping volume.

2. An apparatus as claimed in claim 1 wherein either or both probes is a mechanical 3-D probe or a multidimensional probe.

3. An apparatus as claimed in claim 1, wherein the first and second probes are curved.

4. An apparatus as claimed in claim 1, wherein the first and second probes are angled towards the propagation axis.

5. An apparatus as claimed in claim 1, wherein the mount is a housing that houses the probes and the medical instrument guide is a channel extending through the housing between the probes.

6. An apparatus as claimed in claim 1, wherein the mount is a plate and the medical instrument guide is a channel extending through the plate between the probes.

7. An apparatus as claimed in claim 1, wherein the medical instrument guide is detachably mountable to the mount in one or more orientations.

8. An apparatus as claimed in claim 1, wherein the medical instrument guide comprises means for tracking the position of the medical instrument guide relative to the probes.

9. An apparatus as claimed in claim 1 further comprising a third ultrasound probe, configured to acquire a 3-D image of a third volume, and mounted on the mount such that the first, second and third volumes overlap to form the overlapping volume.

10. An apparatus as claimed claim 1, wherein the mount is a member and the probes are mounted to the member such that a space is provided between the probes for location of the medical instrument guide therein.

11. A method of using of the apparatus as claimed in claim 1, in an epidural anaesthetic procedure, comprising
placing the apparatus over a back of a patient such that the medical instrument guide is placed over a needle insertion point on the back, and
emitting an ultrasound signal into the back and capturing images of the first and second volumes, wherein the images of the first and second volumes include a section of a patient's spine.

12. A method as claimed in claim 11 wherein the target is an epidural space.

13. A method as claimed in claim 12 wherein each probe is placed at a paramedian location with respect to the spine.

14. A method as claimed in claim 13 wherein each probe is placed over spinae erector muscles of the patient.

15. A method as claimed in claim 11 further comprising inserting a needle through the medical instrument guide and along the propagation axis that intersects the target, such that the captured images includes an image of the needle.

16. An ultrasound imaging and medical instrument guiding apparatus, comprising:
a first ultrasound probe configured to acquire a 2-D image of a first plane;
a second ultrasound probe configured to acquire a 3-D image of a first volume,
a mount on which the first and second probes are mounted,
the first and second probes located on the mount such that the first volume intersects the first plane; and
a medical instrument guide positionable relative to the first and second ultrasound probes and configured to receive and guide a medical instrument along a propagation axis to a target such that the target and the propagation axis intersect the first volume and first plane.

17. A system for acquiring and displaying ultrasound medical images, comprising:
an ultrasound imaging and instrument guiding apparatus as claimed in claim 1;
circuitry communicative with the ultrasound imaging and instrument guiding apparatus to receive the first and second volumetric datasets therefrom and comprising a processor with a memory having programmed thereon steps and instructions for execution by the processor to:
condition the first and second volumetric datasets;
combine the first and second volumetric datasets and calculate the overlapping volume in the first and second volumetric datasets;
perform one or both of ray-tracing and re-slicing to produce one or more 2-D images from the overlapping volume;
enhance the one or more of the produced 2-D images; and
a display device communicative with the circuitry to receive and display one or more of the produced 2-D images.

18. A system as claimed in claim 17 wherein calculating the overlapping volume comprising spatial compounding.

19. A system as claimed in claim 15, wherein ray-tracing is performed to produce a 2-D projection image of one of the first and second volumetric datasets, or of a combination of the first and second volumetric datasets.

20. A system as claimed in claim 15, wherein re-slicing is performed on the first or second volumes or the calculated overlapping volume to produce a cross-sectional plane image.

21. A system as claimed in claim 15, wherein the memory is further programmed to calculate an anticipated trajectory of the needle along the propagation axis, and overlay the calculated anticipated trajectory on one or more of the produced 2-D images.
